(19) United States
(12) Patent Application Publication
(10) Pub. No.: US 2021/0050118 A1
(43) Pub. Date: Feb. 18, 2021

(54) Systems And Methods For Facilitating Expert Communications

(71) Applicant: Noetic Insights, LLC, Morristown, NJ (US)

(72) Inventors: Richard Goldstein, Whitehouse Station, NJ (US); Jeffrey W. Meehan, Mendham, NJ (US); Sanjiv Agarwala, Center Valley, PA (US)

(21) Appl. No.: 16/991,188

(22) Filed: Aug. 12, 2020

Related U.S. Application Data
(60) Provisional application No. 62/886,053, filed on Aug. 13, 2019.

(51) Int. Cl. G16H 80/00; H04L 29/06; G06F 3/0482; H04L 29/08; G06N 3/08

(52) U.S. Cl. CPC G16H 80/00; H04L 65/1069; G06N 3/08; G06F 3/0482; H04L 67/36; H04L 65/1089

(57) ABSTRACT

A communication system and method including displaying a request screen, the request screen including an area for selecting one or more diagnoses or entering a name of an illness and a submit indicator; generating a request in response to a requestor selecting the submit indicator on the request screen; identifying one or more other users as eligible users eligible to provide content in response to the request, the identifying including matching respective records of the one or more other users to the request; sending out invitations to one or more of the eligible users; and displaying a session screen, the session screen corresponding to a communication session and including an indicator for each of one or more participants in the communication session, wherein the one or more participants includes the requestor and one or more eligible users who have accepted an invitation.

### CROSS-REFERENCE TO RELATED APPLICATION

The present application claims the benefit of the filing date of U.S. Provisional Patent Application No. 62/886,053, filed on Aug. 13, 2019, the disclosure of which is hereby incorporated herein by reference.

### BACKGROUND

The advent of computer communications and smartphones has allowed users of such devices to readily submit requests for information to a network of other users. However, in order to ensure that the number of responses to a user request are manageable and pertinent, it is necessary for the user to carefully select destinations for the requests and carefully review the responses. Moreover, requests sometimes include solicitations for medical opinions, and one or both of a requestor and responders may wish to remain anonymous, which further complicates the provision of requests and responses.

### BRIEF SUMMARY

In view of the need to efficiently provide networked communication of requests and responses, and the desire for anonymous requests and responses, the present technology is provided.

In accordance with an aspect of the technology a communication method includes displaying a request screen, the request screen including an area for selecting one or more diagnoses or entering a name of an illness and a submit indicator; generating a request in response to a requestor selecting the submit indicator on the request screen; identifying one or more other users as eligible users eligible to provide content in response to the request, the identifying including matching respective records of the one or more other users to the request; sending out invitations to one or more of the eligible users; and displaying a session screen, the session screen corresponding to a communication session and including an indicator for each of one or more participants in the communication session, wherein the one or more participants includes the requestor and one or more eligible users who have accepted an invitation.

In accordance with an aspect of the technology a communication system includes a display; and one or more processors for controlling displaying a request screen on the display, the request screen including an area for selecting one or more diagnoses or entering a name of an illness and a submit indicator, generating a request in response to a requestor selecting the submit indicator on the request screen, identifying one or more other users as eligible users eligible to provide content in response to the request, the identifying including matching respective records of the one or more other users to the request, sending out invitations to one or more of the eligible users, and displaying a session screen on the display, the session screen corresponding to a communication session and including an indicator for each of one or more participants in the communication session, wherein the one or more participants includes the requestor and one or more eligible users who have accepted an invitation.

In accordance with an aspect of the technology a non-transitory computer-readable medium stores instructions for implementing a communication method, the method including displaying a request screen, the request screen including an area for selecting one or more diagnoses or entering a name of an illness and a submit indicator; generating a request in response to a requestor selecting the submit indicator on the request screen; identifying one or more other users as eligible users eligible to provide content in response to the request, the identifying including matching respective records of the one or more other users to the request; sending out invitations to one or more of the eligible users; and displaying a session screen, the session screen corresponding to a communication session and including an indicator for each of one or more participants in the communication session, wherein the one or more participants includes the requestor and one or more eligible users who have accepted an invitation.

### BRIEF DESCRIPTION OF THE DRAWINGS

The foregoing aspects, features and advantages of the present disclosure will be further appreciated when considered with reference to the following description of exemplary embodiments and accompanying drawings, wherein like reference numerals represent like elements. In describing the exemplary embodiments of the present disclosure illustrated in the drawings, specific terminology may be used for the sake of clarity. However, the aspects of the present disclosure are not intended to be limited to the specific terms used.

FIG. 1 is a functional block diagram of an example system according to an embodiment.

FIG. 2 shows a request screen that may be displayed on a user's device according to and embodiment.

FIG. 3 shows a session screen that may be displayed on a user's device according to an embodiment.

### DETAILED DESCRIPTION

A system and method for facilitating the exchange of information between users, particularly in the case of identifying experts that can assist patients and other entities with medical requirements in connection with a medical diagnosis, is disclosed herein.

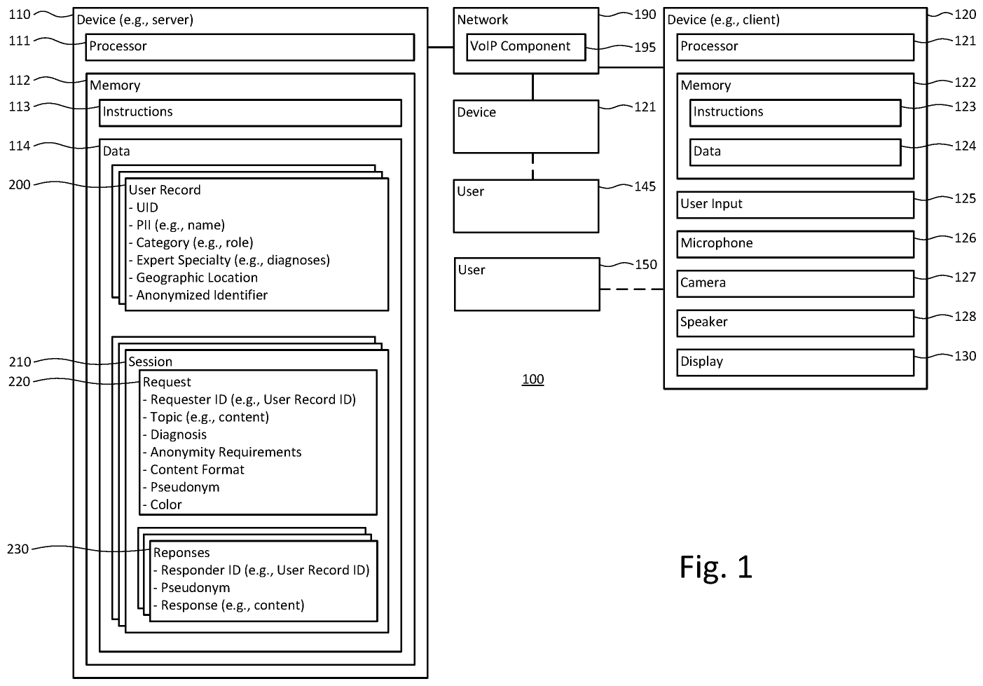

Systems such as those described above may include one or more computing devices. For instance, FIG. 1 provides the example of system 100, which includes computing devices 110, 120 and 121. The computing devices are configured to accept information, perform operations based on that information, and take an action or provide additional information in response. The computing devices may be, or include, a processor that is capable of receiving one or more electrical signals representing information expressed as a numerical value as input, determine a numerical value based on the input in accordance with instructions, and provide one or more electrical signals that represent the determined numerical value as output. Device 110 includes processor 111, which may be a commercially available central processing unit (CPU), application-specific integrated circuit (ASIC) or field-programmable gate array.

The instructions used by a computing device include any set of one or more instructions that are accessed and executed by the computing device. By way of example, device 110 stores values representing instructions 113 and processor 111 is able to access those values and perform, or cause other components of device 110 or system 100 to automatically perform, operations associated with those instructions. Instructions 113 may be stored in a format that is capable of execution by processor 111 with or without additional processing, e.g., machine code, object code, script, or independent source code modules that are interpreted on demand. An operation expressed as a single instruction in one format may correspond with multiple instructions in another format, e.g., executing a single command in script may require the execution of multiple machine code instructions. Some of the operations described herein may involve the execution of instructions provided by an operating system.

The instructions may be stored in a memory. For instance, instructions 113 are stored in memory 112. The memory may be any component that is capable of storing information on a non-transitory storage medium that can be read by a computing device, e.g., registers provided by processor 111, volatile memory such as RAM (random-access memory), non-volatile memory such as flash memory (e.g., a Secure Digital (SD) card), a hard-disk drive, a solid-state drive, optical storage, or tape backups. Device 110, processor 111 and memory 112 are configured so that processor 111 can read, modify, delete and add values stored in memory 112. Memory may be configured to provide less access than the example of memory 112, e.g., the memory may be read-only.

Memory may store information that is used by, or results from, the operations performed by the computing device. By way of example, memory 112 stores data 114, which includes values that are retrieved or stored by processor 111 in accordance with instructions 113, such as information that is required or determined by device 110 when performing some of the operations described herein. Values stored in memory 112 may be stored in accordance with one or more data structures. For instance, a value stored in memory 112 may represent a single numeric value (e.g., a binary number, an integer, a floating point number, a Unicode value representing a single character of text (such as a letter, digit or punctuation mark), or a value representing a single machine code instruction), a set of multiple numeric values (e.g., an array of numbers, a string of text characters, XML-formatted data, or a file), or information from which values to be processed in accordance with instructions 113 may be obtained (e.g., a reference to a value stored at a remote location or a parameter of a function from which the required value is calculated).

A computing device may include components for receiving information from the physical environment surrounding the device to allow direct user input to the device. Similar to device 110, device 120 includes a processor 121, memory 122, instructions 123 and data 124. Device 120 also includes components that detect information relating to the physical environment in which the component is disposed, and this information may include information provided by user 150. Device 110 includes a user input component 125 having circuitry and other components configured to receive input from user 150, such as information provided tactilely (e.g., a mouse, keyboard, keypad, button or touchscreen). User input components may perform functions that are not primarily directed to user input. By way of example, camera 127 may be used to capture user commands (e.g., hand gestures) and other visual information (e.g., the visual characteristics of a person's body). Microphone 126 may be used to capture user commands (e.g., verbal commands) and other audio information (e.g., the sound of a heartbeat).

A computing device may include components for providing information via the physical environment surrounding the device to provide output directly to users. For example, a component may include circuitry that outputs visual, audio or tactile (e.g., haptic) information to users of the device, such as display 130 (e.g., a computer monitor, a touch-screen, a projector or another component that is operable to change a visual characteristic in response to a signal), speaker 128, or an actuator to vibrate the device.

A computing device may include one or more components for communicating with other computing devices. By way of example, devices 110, 120 and 121 include circuitry (e.g., a network interface) connecting each device to a different node of communication network 190. Network 190 may be composed of multiple networks using different communication protocols. For instance, when device 110 transmits information to device 120, the information may be sent over one or more of the Internet (e.g., via core Internet routers in accordance with the Transmission Control Protocol (TCP) and Internet Protocol (IP)), a cellular network (e.g., in accordance with the LTE (Long-Term Evolution) standard), a local network (e.g., an Ethernet or Wi-Fi network), or a Bluetooth connection. A device may provide information to a user via other devices, e.g., device 110 may display information to user 150 by sending the information via network 190 to device 120 for display on display 130. A computing device may also provide information to another computing device without the use of a network. Although only a few computing devices are depicted in FIG. 1, the system may include a large number of computing devices that are connected to the network at a large number of nodes.

Although FIG. 1 shows computing devices 110 and 120 as individual blocks, each of which contains its own processor and memory, the operations described herein may involve a single computing device or many computing devices, e.g., in the “cloud”. For example, various operations described below as involving a single computing device (e.g., a single central processing unit (CPU) in a single server) may involve a plurality of computing devices (e.g., multiple processors in a load-balanced server farm or otherwise having a distributed configuration). Similarly, memory components at different locations may store different portions of instructions 113 and collectively form a medium for storing the instructions. By way of further example, operations described as involving a plurality of computing devices may be performed by a single computing device, e.g., rather than sending data to device 110 for processing, device 120 may process the data itself. Alternatively, device 120 may function as a “thin” client wherein device 110 performs all or nearly all operations that are not directly related to receiving and providing information to user 150 via user input component 125 and display 130. Various operations described herein as being performed by a computing device may be performed by a virtual machine. By way of example, instructions 113 may be specific to a Windows server, but the relevant operations may be performed by a Linux server running a hypervisor that emulates a Windows server.

In many of the examples described herein, device 110 is a server and devices 120-121 are client devices. For instance, device 110 may be a web server and device 120 may be a desktop computer system, e.g., processor 121 and memory 122 may be contained in a desktop personal computer, display 130 may be an external monitor connected to the personal computer by a cable, and user input component 125 may be an external keyboard that communicates with the computer via Bluetooth. Alternatively, device 120 may be a wireless phone with a touchscreen that functions as both display 130 and user input component 125. Other client devices may include, by way of example, laptops, notebooks, netbooks, tablets, and wearable devices (e.g., a smartwatch). In that regard, a computing device may include other components that are typically present in such devices or general purpose computers but are not expressly described herein.

The system may store information associated with users, e.g., each user may be associated with a user record. For instance, data 114 may store user records such as user record 200. Each user record may include a unique user identifier (UID) that is unique to each user record.

A user record may also be associated with one or more categories. By way of example, each record may be associated with one or more role categories that identifies the type of information that a user provides to other users via system 100. For instance, user record 200 may identify the user associated with the record as a requester (e.g., someone requesting information from other users), an expert (e.g., someone who provides information in response to requests from other users), or a service provider (e.g., an administrator or customer service representative associated with the entity operating server 110). A user record may have multiple role categories, each one of which depends on additional data. By way of example, a user may serve the role of an expert in one session and the role of a requester in another session.

Each user record may also be associated with personally-identifiable information (PII). For instance, if the user associated with user record 200 is a natural person, the PII may comprise the user's first and last name, residence address, or other information by which the individual associated with the user record may be identified. PII may further comprise a photo of the person. If the user associated with a particular user record is an entity other than a natural person, such as a corporation or other legal entity, then PII may include, by way of example, the corporation's or entity's legal name, office address, or the like. For instance, user record 200 may alternatively be associated with a drug company. In some aspects of the system, whether certain categories of particular information associated with a user are considered PII may depend on criteria set by the user, a service provider, or third parties. For example, system 100 may be configured to automatically designate certain types of information as PII by default and permit a user to designate certain types of information as PII or not PII by default.

A user record may also be associated with one or more specialties. For example, user record 200 may indicate that the user is an expert in pulmonology. In that regard, the specialty data stored in connection with user record 200 may be represented by a single enumerated value identifying a specific profession chosen from a list curated by the operators of a website, one or more ICD diagnosis codes, or a string of words that the user chose for himself or herself.

Each user record may be further associated with geographic location data. The type and scope of the location data may depend on the nature of the information represented by the data. For instance, if the record is associated with a medical doctor, the geographic information may include both the specific street address of the doctor's office and the countries and state(s) from which the doctor has received a medical license. Certain types of location information, such as an office address, may be considered PII.

A user record may also include contact information. For example, user record 200 may include data identifying the method by which the user associated with user record 200 would like to receive automatic notifications from system 100, e.g., an email address, a phone number to receive texts, etc. Contact information may be considered PII.

User records may be associated with additional information as indicated in more detail below.

The system may store data relating to sessions. As indicated by session data 210 in FIG. 1, a session record may be associated with a request from a user relating to a topic and data received from other users (or the original user relating to the request) in response to the request.

For instance, users may submit requests for information from other users regarding a topic selected by the requester. Among other things, the topic may be a question about a diagnosis, e.g., the requester may be a person who has been diagnosed with cancer and would like more information about his or her diagnosis. Alternatively, the requester may be a doctor or hospital who would like suggestions for alternative treatments if a currently prescribed therapy is not working as well as hoped. Yet further, the requester may be a company which would like more information regarding doctors' experiences with a specific drug in connection with a specific diagnosis. In that regard, request record 220 may identify both the user record of the user that submitted the request and the content describing the topic.

Content describing a topic of a request, and the content submitted by other users in response, may be provided and stored in a variety of different data formats. For instance, content may be provided to server 110 by user 150 as text (e.g., a string of words or characters) that were typed by user 150 using a keyboard.

Alternatively or in addition, user 150 may provide content to server 110 as audio data. By way of example, if the session is being conducted via a real-time teleconference and device 120 is a cell phone, user 150 and others users may submit content by using device 120 to call a phone number associated with VoIP component 195 (e.g., a component provided by a VoIP service provider). Alternatively, user 150 may also provide audio content by using microphone 126 to store an audio file and upload the file to server 110.

Content may be submitted in formats other than strictly text or strictly audio. By way of example, the content may also be visual but non-textual (e.g., images of scans of the user or other patients), combinations of audio and image data (e.g., a live audio/visual conference that captures the response of the user during a real-time video conference) or tactile (e.g., haptic patterns).

A request record may define and limit the types of data formats that may be submitted in connection with a session. For instance, the user that submitted the content associated with request record 220 may require all responses to be submitted in real-time during a teleconference among the requester and experts providing a response.

A request record may also be associated with parameters that indicate whether PII associated with a user record will be available for access by other participants. By way of example, request record 220 may indicate that the PII of one (e.g., just the requester), some (e.g., the responder but not the requester) or all (e.g., a double blind discussion where the requester and all of the responders are anonymous) will not be displayed or otherwise accessible to the other participants of the session.

While the technology disclosed herein is not limited to any type of topic, the technology disclosed herein is particularly advantageous in connection with requests for anonymous medical advice. By way of example only, a user with a challenging illness may have questions about their treatment but may not want to disclose their name or address for privacy reasons. Similarly, a person may be willing to submit a response to a request based on the information that was provided by the requester, but he or she may be less willing to provide a response if doing so exposes his or her PII to the requester. For example, an expert may believe that he or she has the time and experience to answer new questions but may not have the time to respond to additional questions after the initial question-and-answer session is over. Advice from experts may be stored in data 114 as response records 230, wherein each response record identifies the user ID of the responder, the content of the response and, if applicable and as explained below, a pseudonym.

Operations in accordance with a variety of aspects of the method will now be described. It should be understood that the following operations do not have to be performed in the precise order described below. Rather, various steps can be handled in different order or simultaneously.

A user may solicit information from other users regarding a topic by logging into the system and submitting a request. For instance, if user 150 is the user associated with user record 200, user 150 may use device 120 to log into a website hosted by server 110 via network 190 by providing his or her user ID, password or other authentication information.

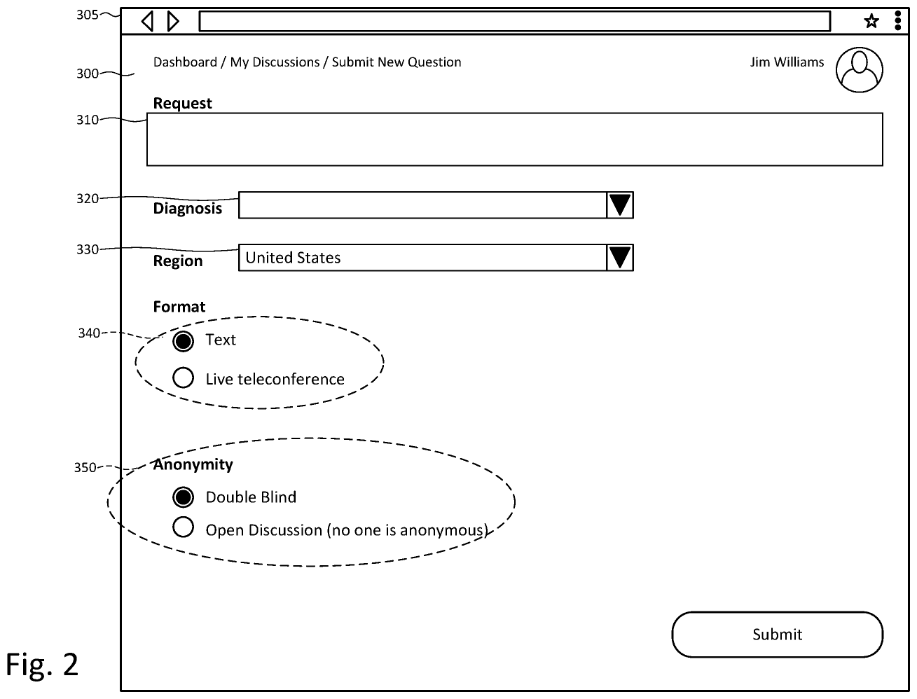

A user may submit a request by providing content relating to a topic. For instance, upon user 150 being authenticated by the site hosted by server 110 and the user providing an indication that they want to submit a request for a text-based discussion, server 110 may prompt user 150 to enter request related information via webpage 300 shown in FIG. 2. The webpage 300 may be provided by the server 110 and displayed by running browser 305 stored on device 120. In that regard, user 150 may enter the content of the topic via textbox 310.

A request may identify one or more diagnoses. For instance, user 150 may select from an enumerated list as shown by the dropdown list 320 shown on webpage 300 (e.g., ICD-10-CM codes) or type the name of a specific illness.

A request may place limits on the types of content that may be submitted in response. By way of example, user 150 may use radio buttons 340 to select between text-based responses or a live teleconference. In the example shown in FIG. 2 and certain other figures, it is assumed user 150 selected text. If the user selected live teleconference, webpage 300 may prompt the user to enter a proposed starting date and time. In other aspects of the technology described herein, user content may be submitted in other formats as well. By way of example, the system may permit audiovisual user content (e.g., video files) to be submitted.

The requester may also select a geographic region (e.g., country, state(s) or any other region or set of geographic regions) related to their request. For instance, the requester may be primarily interested in the opinions of doctors who practice in the United States.

A requester may also request that the system automatically anonymize certain aspects of the information contained in the user records of the participants. By way of example, user 150 may use radio buttons 350 on webpage 300 to make the session double blind (in which case PII relating to both the requester and responders is anonymized during the session) or open (in which case some of the PII relating the requester and responders is available). In other aspects of the system, each participant in the session may choose to anonymize their information or not, and may have control over which types of PII is available for access by other participants.

Before a new session is started, the system may query data 114 for sessions that contain content that is similar to the content of the request. For instance, system 100 may use natural language processing and machine learning to determine whether a session record 210 is sufficiently likely to be of interest to the requester. If such a session record is found, system 100 may display the content of the session to the user and ask the user to indicate they still want to start a new session.

The system may identify users based on the request. For instance, in response to receiving a request, processor 111 may query data 114 to identify any user record 200 that is associated with information indicating that the record's associated user is eligible to provide content in response to the request.

For instance, a user record may be considered eligible if one or more of the record's specialties matches a diagnosis provided with the request. By way of example, if both the request's diagnosis and a record's specialties are stored in memory 112 as a single ICD-10-CM code, the processor may automatically designate a record as eligible if the diagnosis code matches the specialty code exactly, or if the user record's specialty code is a category that contains the diagnosis as a subcategory.

The system may also designate a user record as eligible based on an estimate of the likelihood of the record's user providing useful content in response to the request. In that regard, processor may calculate, for each record, a score that is related to such likelihood and based on one or more parameters.

One such parameter may reflect the extent to which the request's diagnosis matches the user record's specialties. By way of example, if a diagnosis of the request is an ICD-10-CM code and that code is identical to a code listed as a record's specialty, the processor may assign a value of 1.0 to a diagnosis/specialty match parameter. The processor may assign a value of 0.5 to the same parameter if the ICD-10-CM code of the diagnosis is a subcategory of an ICD-10-CM code listed as a record's specialty, e.g., the record's listed specialty includes but does not specifically identify the diagnosis.

Another parameter may reflect the extent to which the request's geographic area of interest overlaps with, or is proximate to, a location specified by the records geographic location data. For example, processor 111 may assign a value of 1.0 to a geographic match parameter if both a user's current office location and a region from which the user received a medical license are contained in the request's geographic area of interest, and may assign a value of 0.5 to the same parameter if a region from which the user received a medical license is contained in the area of interest but the user's office is not. The processor may also assign a value of 0.1 to the geographic match parameter if none of the record's locations are contained in the area of interest but one or more those locations border, or are within a threshold distance of, the request's area of interest. Otherwise, the processor may otherwise assign a value of 0.0 to the geographic matching parameter.

User records may also be scored based on other parameters. By way of example, a parameter may be based on information that is dependent on information associated with the user record but independent of information associated with the request (e.g., how frequently the user has accepted invitations to respond to other requests, or the historical record of the ratings that a user record has received in response to prior requests). Another parameter may be based on information that is independent of both the user record and the request, e.g., a randomly generated number. Moreover, each of the aforementioned parameters may actually comprise multiple different parameters. For instance, instead of a single geographic match parameter, one parameter may be associated with a record's listed office location and a different parameter may be associated with the record's list of regions that issued a medical license to the associated user.

The system may determine whether a user record is eligible to provide content in response to a request based on a comparison of one or more of the parameter values to thresholds. The processor may calculate a score that is based on the sum of parameters, e.g., the value of the diagnosis/specialty match parameter plus the value of the geographic match parameter. A user may record may be considered eligible if its score exceeds a threshold value.

The system may further assign different weights to different parameters. For example, processor 111 may calculate the score based on the sum of the value of the diagnosis/specialty match parameter multiplied by a first weight value and value of the geographic match parameter multiplied by a second weight value, wherein the first weight value is significantly greater than the second weight value. The weights may be static or dynamically determined, e.g., based on priorities provided with the request. For instance, request entry screen 300 may allow the requester to prioritize certain parameters, e.g., the requester may indicate that geographic location is very important, in which case the second weight value may be set greater than the first weight value.

Records may be designated as eligible or ineligible based on both the score and other factors. By way of example, processor 111 may automatically designate a user record as ineligible if there is no overlap between the request's diagnosis and the record's specialties.

Processor 111 may automatically designate a user record as ineligible, or assign negative weight to a user record specific parameter, if the request contains information that contradicts eligibility. For instance, user record 200 may include data that identifies entities from whom the associated user has accepted or provided grant money (with or without regard to a particular diagnosis or drug). The system may permit the requester to automatically designate as ineligible, or weight negatively, a user record if the record indicates the user has accepted or provided grant money to or from the requester. In that regard, the system 100 may determine that certain users are not eligible to respond to a request on the basis of their PII even if the requester designated the request as double blind.

In addition or alternatively, user records may also be identified based on machine learning. For instance, each time a session is concluded, a neural network may be further trained with the user records associated with the experts, the content of the request, and ratings provided by the requester in response. When a new request is received, the content of the request may be used as input to the neural network, and the output may comprise a user record that the neural network determines is the most likely to be highly rated by the user.

After the system identifies eligible records, the system may automatically send invitations to the records' associated users to submit content in response to the request. For instance, system 100 may use an eligible record's notification information to send the user an email or text (SMS) regarding the request. Such an email may contain, or contain a link to, one or more of the diagnosis, the content of the request itself, the geographic area of interest, anonymity, start time if the session is conducted in real time, and other information. A user may also be notified of pending invitations each time they log into system 100 via a browser or dedicated app (e.g., via a pop-up). If and to the extent a portion of system 100 is implemented as a dedicated app on a mobile device, the notification may also be provided as a push notification from the app. A user may also log into server 110 and request that the system check whether his or her user record is eligible to receive any invitations to pending requests.

Users may provide the system with an indication of whether they accept or reject an invitation to submit content. By way of example, an email invitation may contain a hyperlink that says “accept”. The link may contain a URL hosted by server 110, and the link may further contain CGI parameters identifying a unique ID assigned to the request and a unique ID assigned to the user. Clicking the link may automatically launch and navigate a browser to an authentication page hosted by server 110 that prompts the user to enter their user name and password. Upon being authenticated and as explained in more detail below, server 110 may then navigate the browser to a page where the user can view the request and enter content in response. Users may also provide the system with an indication of whether they decline the invitation. For instance, an email may contain a “reject” hyperlink that is similar to the “accept” hyperlink discussed above, but results in the user rejecting the invitation.

The system may automatically withdraw invitations if they are not accepted within a given amount of time after the notification was sent, displayed or otherwise made available for access by a user. In that regard, the system will no longer include a user in a session if the user attempts to accept an invitation that has been withdrawn.

The system may send invitations to less than all of the eligible records' users of the relevant request. For instance, the system may rank the eligible records based on the aforementioned score and only notify a given number of the top-scoring eligible records.

The given number of invited participants may depend on the minimum and maximum number of users that are desired to participate in the session associated with the request. The minimum and maximum numbers may be statically or dynamically determined and represent preferences or absolute limits. By way of example, the minimum and maximum numbers may reflect a static range selected by the system provider that indicates a session will not start unless and until at least two users accept an invitation to participate and that all unaccepted invitations are automatically withdrawn once seven users have accepted their invitations to the session. The minimum and maximum numbers may also be the same number and reflect a preference rather than a requirement, e.g., the requester may indicate that the optimal number of participants (excluding the requester) is three. The minimum and maximum number may also be dynamically determined by the system. For instance, the system may automatically determine (e.g., based on data analysis of requester feedback), that requesters in some geographic regions generally prefer to have less participants than requesters from other geographic regions. The given number may also be based on a statistical analysis of how many invitations typically need to be sent in order to obtain a quantity of acceptances that falls within the minimum and maximum range.

The system may automatically send and withdraw groups of invitations in stages. When the system sends the given number of invitations to the users of the highest-ranked user records, the system may periodically determine how many of the invited users in that group have responded. If the system determines that less than a minimum number of acceptances has been received within a given amount of time (e.g., a few hours), the system may automatically withdraw any unaccepted invitations in that group. The system may thereafter send the given number of invitations to a second group of users, e.g., the users associated with the highest-scoring user records that are ranked below the prior group. The process of sending groups of invitations may continue until the minimum number of participants have accepted invitations to join a session discussing the content.

Sending invitations as described above is believed to overcome or mitigate a number of technical problems associated with platforms that automatically generate invitations. For example, given the nature of push notifications like emails and text messages, it is often not possible to know whether a failure to respond reflects a user's implicit rejection of the content or the fact that the fact that the user is busy with other matters. This is particularly so when the users are medical doctors who may be too busy with other patients to review or respond to every invitation within a few hours. Yet further, it is often difficult to predict how many invitations will be accepted, which makes it difficult to predict how many invitations need to be sent to reach the minimum number of participants without exceeding the maximum number. For instance, it is often necessary to invite more than the maximum number of potential responders in order to timely obtain the minimum number. However, if a request quickly reaches the required number of acceptances, it can be frustrating to busy invitees to routinely and timely accept an invitation but then learn that the session is closed because less busy invitees accepted their invites faster. It can also be suboptimal if sessions reach the maximum number before users have a chance to review invitations associated with highly-scored user records. The system and methods described herein is believed to address and mitigate many of these problems.

Before sending responses to another stage, the system may send a notification to the requester and prompt the requester to indicate whether the currently accepted invitations are acceptable. If the requester indicates that they are acceptable, the system may then start a session.

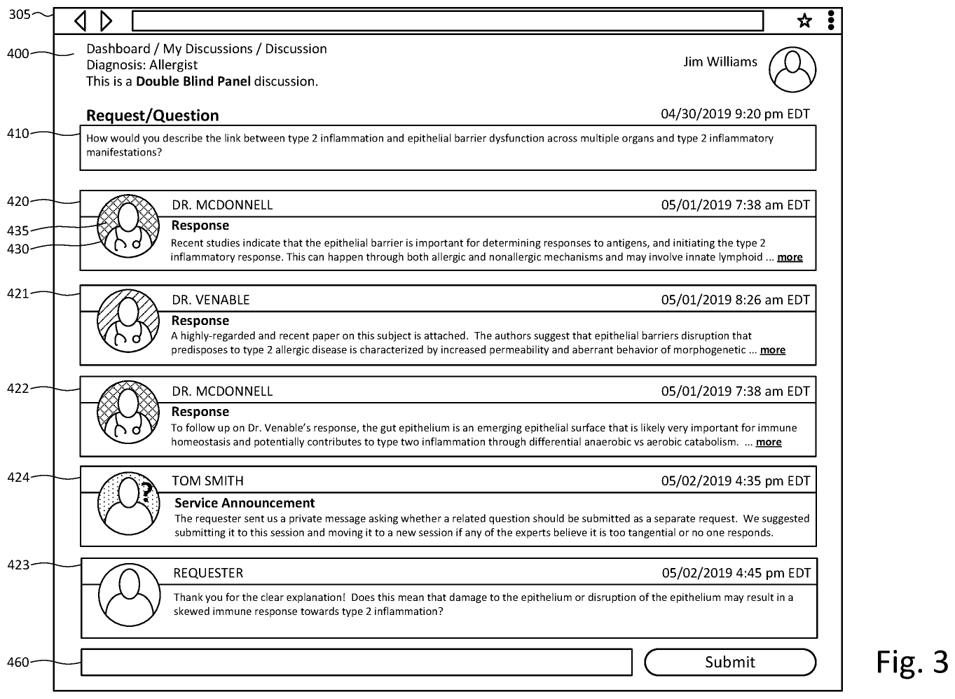

FIG. 3 provides an example of a text-based session. Server 110 may transmit session webpage 400 to device 120 for display on browser 305 to users (such as user 150) that permits the entry of text. For example, portion 410 of webpage 400 may display the text 410 associated with the request content submitted by user 150, and responses 420-422 received from other users in response. The session shown in FIG. 3 also shows a follow up question 423 from the requester and additional information 424 from a service provider. Webpage 400 may also include textbox 460 for entering new content. Participants in a session may be required to limit the content they provide to information that is related to the request.

When displaying, playing or otherwise rendering content submitted by one user to another user during a session, the system may anonymize one or more aspects of the information contained in a user record or content. By way of example, if a session has double-blind anonymity, the system may not display the PII stored in a user record to any other user during or outside of a session.

When anonymizing information associated with a user, the system may determine and associate each participant with a pseudonym that is specific to the session. For instance, if user 145 is an expert that accepted an invitation to submit content in response to the request entered by user 150, system 100 may generate a pseudonym for both user 145 and user 150. The pseudonym may be dependent, at least in part, on information associated with the request or a user record. By way of example, to the extent it does not compromise requested anonymity, the system may randomly select a name that is among the most popular of the given or family names in the geographic area of the requester's area of interest or the responder's geographic location information. The pseudonym may also be independent of data that is associated with the request or user records, e.g., the pseudonym may be randomly selected from the most popular names in the world or generated by a function that randomly concatenates consonants and vowels. If user 145 accepts an invitation to a second session, the system may store a different pseudonym in the user's user record for use during the second session.

Generic sounding and random pseudonyms may impede some user's comprehension of the content. To overcome this potential issue, each participant may also be assigned visual indicia that is specific to the participant during the session. For instance, the visual indicia may include icon 430. The appearance of the icon may be determined based on information contained in the user's applicable user record, e.g., their role category such as using a simple profile for requesters, a profile with a stethoscope for expert responders, and profile with a question mark for service providers. The visual indicia may also be determined based on information that is specific to the session and specific to the participant, but unrelated to a user record. For example, the color of the background 435 of icon 430 may be randomly assigned to user 145 at the beginning of the session and retained throughout the session.

As noted above, sessions may also be voice-based, such as a teleconference. At least some of the users of the system may be well known in their field and recognizable by their voice. In order to facilitate anonymization, system 110 may mask participant's voices during a teleconference by applying a vocoder. The parameters used by the vocoder may be based on information contained in a user's record. For instance, on average, men tend to speak at a lower pitch than women. The vocoder may select parameters that modulate a user's voice a little higher or a little lower in pitch depending on whether (and if) the relevant user record indicates the user is a man or women, respectively. The parameters may also be adjusted dynamically based on the audio signals received during a teleconference.

Sessions may comprise combinations of the different types of content. By way of example, users may exchange text-based content via a screen that looks like webpage 400 during a teleconference-based session. A user may also upload audio files along with their text, such as a recording of the sound of his or her heartbeat, for playing to other users. Yet further, the system may convert content in one format into content into another format, such as by using text-to-speech or speech-to-text.

When a session is completed, each participant may be given an opportunity to rate the quality of another's content. As noted above, the system may use the ratings when selecting user records during the invitation process.

Embodiments of the present technology include, but are not restricted to, the following.

(1) A communication method including displaying a request screen, the request screen including an area for selecting one or more diagnoses or entering a name of an illness and a submit indicator; generating a request in response to a requestor selecting the submit indicator on the request screen; identifying one or more other users as eligible users eligible to provide content in response to the request, the identifying including matching respective records of the one or more other users to the request; sending out invitations to one or more of the eligible users; and displaying a session screen, the session screen corresponding to a communication session and including an indicator for each of one or more participants in the communication session, wherein the one or more participants includes the requestor and one or more eligible users who have accepted an invitation.
(2) The communication method according to (1), wherein the communication session is at least partially anonymous such that the identity of at least one of the participants is not displayed or otherwise accessible to the other participants.
(3) The communication method according to (2), wherein the communication session is a double blind communication session such that the identity of each participant is not displayed or otherwise accessible to each of the other participants.
(4) The communication method according to (1), wherein identifying one or more other users as eligible users includes determining whether respective specialties of one or more of the other users matches at least one of the diagnoses or the name of an illness.
(5) The communication method according to (4) wherein the one or more diagnoses are designated by respective first codes and the specialties of the one or more of the other users are designated by respective second codes, and determining whether respective specialties of one or more of the other users matches at least one of the diagnoses includes determining whether any of the first codes match any of the second codes.
(6) The communication method according to (5), wherein the first codes and the second codes are International Classification of Disease (ICD) codes.
(7) The communication method according to (1), wherein the request screen further includes an area for specifying a geographic area of interest as part of the request, and wherein identifying one or more other users as eligible users includes determining whether respective office locations of one or more of the other users matches a geographic area of interest specified in the request screen.
(8) The communication method according to (1), wherein the request screen further includes an area for specifying a geographic area of interest as part of the request, and wherein identifying one or more other users as eligible users includes determining whether respective areas in which one or more of the other users is licensed matches a geographic area of interest specified in the request.
(9) The communication method according to (1), wherein identifying one or more other users as eligible users includes using content of the request as input to a neural network that has been trained according to previous requests, and receiving as output of the neural network an indication of the eligible users.
(10) The communication method according to (1), wherein sending out invitations to one or more of the eligible users includes sending out invitations to less than all of the eligible users.
(11) The communication method according to (10), wherein the method further includes withdrawing unaccepted invitations and sending out additional invitations after withdrawing unaccepted invitations.
(12) The communication method according to (1), wherein the communication session includes voice communication from at least one of the participants, and wherein the voice communication is masked.
(13) The communication method according to (12), wherein the wherein the voice communication is masked by a vocoder using parameters that are selected based on a gender of the at least one participant.
(14) The communication method according to (1), wherein the session screen includes respective response areas for participants, and each response area is operable to display a response to the request.
(15) The communication method according to (14), wherein the session screen further includes an area for displaying additional information from a service provider.
(16) The communication method according to (14), wherein the session screen further includes, for each participant, a pseudonym specific to the communication session.
(17) The communication method according to (15), wherein the session screen further includes, for each participant, visual indicia specific to the communication session and based on a specialty of the participant.
(18) The communication method according to (1), wherein the request screen and session screen are displayed on a mobile device.
(19) A communication system including a display; and one or more processors for controlling displaying a request screen on the display, the request screen including an area for selecting one or more diagnoses or entering a name of an illness and a submit indicator, generating a request in response to a requestor selecting the submit indicator on the request screen, identifying one or more other users as eligible users eligible to provide content in response to the request, the identifying including matching respective records of the one or more other users to the request, sending out invitations to one or more of the eligible users, and displaying a session screen on the display, the session screen corresponding to a communication session and including an indicator for each of one or more participants in the communication session, wherein the one or more participants includes the requestor and one or more eligible users who have accepted an invitation.
(20) A non-transitory computer-readable medium storing instructions for implementing a communication method, the method including displaying a request screen, the request screen including an area for selecting one or more diagnoses or entering a name of an illness and a submit indicator; generating a request in response to a requestor selecting the submit indicator on the request screen; identifying one or more other users as eligible users eligible to provide content in response to the request, the identifying including matching respective records of the one or more other users to the request; sending out invitations to one or more of the eligible users; and displaying a session screen, the session screen corresponding to a communication session and including an indicator for each of one or more participants in the communication session, wherein the one or more participants includes the requestor and one or more eligible users who have accepted an invitation.

As these and other variations and combinations of the features discussed above can be utilized without departing from the claimed subject matter, the foregoing description of the embodiments should be taken by way of illustration rather than by way of limitation. The provision of examples (as well as clauses phrased as “such as,” “e.g.”, “including” and the like) should not be interpreted as limiting the claims to the specific examples; rather, the examples are intended to illustrate only some of many possible aspects. Similarly, references to “based on” and the like means “based at least in part on”.

1. A communication method comprising:
displaying a request screen, the request screen comprising an area for selecting one or more diagnoses or entering a name of an illness and a submit indicator;
generating a request in response to a requestor selecting the submit indicator on the request screen;
identifying one or more other users as eligible users eligible to provide content in response to the request, the identifying comprising matching respective records of the one or more other users to the request;
sending out invitations to one or more of the eligible users; and
displaying a session screen, the session screen corresponding to a communication session and including an indicator for each of one or more participants in the communication session, wherein the one or more participants comprise the requestor and one or more eligible users who have accepted an invitation.

2. The communication method according to claim 1, wherein the communication session is at least partially anonymous such that the identity of at least one of the participants is not displayed or otherwise accessible to the other participants.

3. The communication method according to claim 2, wherein the communication session is a double blind communication session such that the identity of each participant is not displayed or otherwise accessible to each of the other participants.

4. The communication method according to claim 1, wherein identifying one or more other users as eligible users comprises determining whether respective specialties of one or more of the other users matches at least one of the diagnoses or the name of an illness.

5. The communication method according to claim 4, wherein the one or more diagnoses are designated by respective first codes and the specialties of the one or more of the other users are designated by respective second codes, and determining whether respective specialties of one or more of the other users matches at least one of the diagnoses comprises determining whether any of the first codes match any of the second codes.

6. The communication method according to claim 5, wherein the first codes and the second codes are International Classification of Disease (ICD) codes.

7. The communication method according to claim 1, wherein the request screen further comprises an area for specifying a geographic area of interest as part of the request, and wherein identifying one or more other users as eligible users comprises determining whether respective office locations of one or more of the other users matches a geographic area of interest specified in the request screen.

8. The communication method according to claim 1, wherein the request screen further comprises an area for specifying a geographic area of interest as part of the request, and wherein identifying one or more other users as eligible users comprises determining whether respective areas in which one or more of the other users is licensed matches a geographic area of interest specified in the request.

9. The communication method according to claim 1, wherein identifying one or more other users as eligible users comprises using content of the request as input to a neural network that has been trained according to previous requests, and receiving as output of the neural network an indication of the eligible users.

10. The communication method according to claim 1, wherein sending out invitations to one or more of the eligible users comprises sending out invitations to less than all of the eligible users.

11. The communication method according to claim 10, wherein the method further comprises withdrawing unaccepted invitations and sending out additional invitations after withdrawing unaccepted invitations.

12. The communication method according to claim 1, wherein the communication session comprises voice communication from at least one of the participants, and wherein the voice communication is masked.

13. The communication method according to claim 12, wherein the wherein the voice communication is masked by a vocoder using parameters that are selected based on a gender of the at least one participant.

14. The communication method according to claim 1, wherein the session screen comprises respective response areas for participants, and each response area is operable to display a response to the request.

15. The communication method according to claim 14, wherein the session screen further comprises an area for displaying additional information from a service provider.

16. The communication method according to claim 14, wherein the session screen further comprises, for each participant, a pseudonym specific to the communication session.

17. The communication method according to claim 15, wherein the session screen further comprises, for each participant, visual indicia specific to the communication session and based on a specialty of the participant.

18. The communication method according to claim 1, wherein the request screen and session screen are displayed on a mobile device.

19. A communication system comprising:
a display; and
one or more processors for controlling
displaying a request screen on the display, the request screen comprising an area for selecting one or more diagnoses or entering a name of an illness and a submit indicator,
generating a request in response to a requestor selecting the submit indicator on the request screen,
identifying one or more other users as eligible users eligible to provide content in response to the request, the identifying comprising matching respective records of the one or more other users to the request,
sending out invitations to one or more of the eligible users, and
displaying a session screen on the display, the session screen corresponding to a communication session and including an indicator for each of one or more participants in the communication session, wherein the one or more participants comprises the requestor and one or more eligible users who have accepted an invitation.

20. A non-transitory computer-readable medium storing instructions for implementing a communication method, the method comprising:
displaying a request screen, the request screen comprising an area for selecting one or more diagnoses or entering a name of an illness and a submit indicator;
generating a request in response to a requestor selecting the submit indicator on the request screen;
identifying one or more other users as eligible users eligible to provide content in response to the request, the identifying comprising matching respective records of the one or more other users to the request;
sending out invitations to one or more of the eligible users; and
displaying a session screen, the session screen corresponding to a communication session and including an indicator for each of one or more participants in the communication session, wherein the one or more participants comprises the requestor and one or more eligible users who have accepted an invitation.
